(19) United States
(12) Patent Application Publication
(10) Pub. No.: US 2022/0168192 A1
(43) Pub. Date: Jun. 2, 2022

(54) Cosmetic Cleansing Powder

(71) Applicant: Henkel AG & Co. KGaA, Duesseldorf (DE)

(72) Inventors: Marlene Battermann, Asendorf (DE); Sylvia Kerl, Boenningstedt (DE); Heike Schelges, Willich (DE); Sameh Hasan Fares, Hilden (DE)

(21) Appl. No.: 17/539,188

(22) Filed: Nov. 30, 2021

(30) Foreign Application Priority Data
Nov. 30, 2020 (DE) 102020215081.8

(51) Int. Cl. A61K 8/02; A61K 8/86; A61K 8/73; A61K 8/44; A61Q 5/02; A61Q 19/00; A61Q 5/00; A61K 8/92; A61K 8/89; A61K 8/55; A61K 8/46

(52) U.S. Cl. CPC A61K 8/022; A61K 8/86; A61K 8/732; A61K 8/44; A61Q 5/02; A61Q 19/007; A61K 2800/5428; A61K 8/922; A61K 8/89; A61K 8/553; A61K 8/463; A61K 2800/5424; A61Q 5/006

(57) ABSTRACT

A powdered cosmetic cleansing composition which contains specific amounts of at least one anionic surfactant, at least one polyhydric C₂-C₆ alcohol and at least one polysaccharide, and which is dissolved and foamed in contact with water. The present invention further relates to a method of cleansing the human body and/or hair using the powdered cosmetic cleansing composition.

### FIELD OF THE INVENTION

The application describes cosmetic cleansing agents in powder form based on anionic surfactants, specific polyols and polysaccharides, which dissolve and foam in contact with water. The application further describes methods for the preparation of the powders and their use for cleansing the human body and/or hair.

### BACKGROUND OF THE INVENTION

Surfactant-containing products for cleansing the human body and hair have been known for a long time and are mainly sold as foam baths, shower preparations, shampoos and/or oral care compositions in liquid or paste form in suitable packaging. End users take the required amount of product from the packaging during use and dispose of it after emptying. Compared to classic soaps that are sold in solid form, such products offer the user the advantage of easy and quick handling, which is why they dominate the market today. However, this advantage is achieved by accepting certain disadvantages, which are discussed below. In most cases, the packaging of the described products in liquid or paste form consists of non-recyclable plastic, which is a serious problem from an environmental point of view in view of the constantly growing amount of plastic waste.

Another problem is that previous products usually contain higher quantities of water or water/solvent mixtures, which means that the products have a larger volume and, possibly of greater importance from a transport point of view, a relatively high weight. This is disadvantageous for several reasons. In times of increasing water scarcity, resources should be saved. An undesirable, increased transport volume associated with large-volume heavy products is also important from an environmental and cost perspective. Another interesting point is that worldwide travel activity is constantly increasing. Consumers are therefore increasingly interested in cosmetic products that are easy to transport due to their low weight and volume. This is particularly relevant with regard to air travel, as larger containers containing liquids are generally excluded from being carried in an aircraft cabin, so that a passenger travelling only with hand luggage often finds himself in the situation, due to the cosmetics products that dominate the market today, of not being able to take his preferred product selection with him or having to decant the corresponding products into smaller containers first, which, however, is generally accompanied by an even greater volume of packaging material.

### BRIEF SUMMARY OF THE INVENTION

Providing alternative product forms with very low water content, contained in more environmentally friendly, for example recyclable, packaging to save space, is therefore an important goal in the formulation of improved, contemporary and sustainable cosmetic products.

WO 2019/023332 discloses shampoos in powder form containing substantially sulphate-free surfactants (and other surfactants) and thickening and conditioning agents. However, it turned out that the care effect of such products—especially on very dry, stressed skin and/or on dry, damaged hair—was not always completely satisfactory.

Therefore, there is still a need for cosmetic cleansing agents in powder form which, in addition to the aforementioned requirements, also have improved care properties, especially on very dry, stressed skin and/or on dry, damaged hair.

In view of the problems and requirements described above, the inventors have therefore set themselves the task of providing cosmetic cleansing formulations in powder form which can be produced in an energy-saving manner, are easy to use and in which the use of preservatives can essentially be dispensed with.

In addition to satisfactory cleansing, the powder formulations are also said to have increased conditioning benefits on the skin and/or hair.

### DETAILED DESCRIPTION OF THE INVENTION

The task of the invention is solved by the formulations, methods and uses described in detail below.

A cosmetic cleansing composition in powder form containing, based on the total weight of the cleansing composition
a) 10.0 to 70.0 wt. % of at least one anionic surfactant,
b) 1.0 to 8.0% by weight of at least one C₂-C₆ polyhydric alcohol,
c) 20.0 to 40.0% by weight of at least one polysaccharide.

A powder-form cosmetic cleansing agent according to the preceding point, containing—based on the total weight of the cleansing agent—15.0 to 65.0% by weight, preferably 20.0 to 60.0% by weight and particularly preferably 25.0 to 55.0% by weight of at least one surfactant a).

A powder-form cosmetic cleansing agent according to one of the preceding points, containing as surfactant(s) a) amino acid-based surfactants, isethionates and/or alkyl sulphates.

A powder-form cosmetic cleansing agent according to one of the preceding points, containing - based on the total weight of the cleansing agent—1.0 to 7.5% by weight, preferably 1.5 to 7.0% by weight and particularly preferably 2.0 to 6.5% by weight of at least one polyhydric C₂-C₆-alcohol b).

A powder-form cosmetic cleansing agent according to one of the preceding points, containing as polyhydric alcohol b) 1,2-propylene glycol, 1,3-butylene glycol, dipropylene glycol, glycerol and/or diglycerol.

Cosmetic cleansing agent in powder form according to the preceding point, containing as polyhydric alcohol b) glycerol.

A powder-form cosmetic cleansing agent according to one of the preceding points, containing - based on the total weight of the cleansing composition—21.0 to 38.0% by weight, preferably 22.0 to 36.0% by weight and particularly preferably 23.0 to 34.0% by weight of at least one polysaccharide c).

A powdered cosmetic cleansing agent according to one of the preceding points, containing as polysaccharide c) starch fractions from maize, potatoes, rice, wheat and/or tapioca and/or derivatives of starch such as amylose, amylopectin and/or dextrin.

Cosmetic cleansing agent in powder form according to the preceding point, containing starch fractions from maize.

A powder-form cosmetic cleansing agent according to one of the preceding points, additionally containing at least one amphoteric and/or zwitterionic surfactant in a proportion by weight of 0.5 to 10% by weight of the total weight of the cosmetic cleansing agent.

A powder-form cosmetic cleansing agent according to any one of the preceding points, additionally comprising at least one cationic polymer in a proportion by weight of 0.1 to 3% by weight of the total weight of the cosmetic cleansing agent.

A powder-form cosmetic cleansing agent according to the preceding point, comprising at least one cationic polysaccharide polymer, preferably at least one cationic polymer obtainable from cellulose, guar, cassia and/or inulin.

A powdered cosmetic cleansing agent according to any one of the preceding points, additionally containing trimethylglycine (betaine) in a proportion by weight of 0.1 to 3% by weight of the total weight of the cosmetic cleansing agent.

Powdered cosmetic cleansing agent according to one of the preceding points, containing at least one further cosmetic active ingredient and/or excipient, selected from
Lipids,
vegetable oils, butters and/or waxes,
Protein hydrolysates,
Silicones,
Plant extracts and/or
Anti-dandruff active ingredients.

A powdered cosmetic cleansing agent according to any one of the preceding points, which is substantially free from water and/or preservatives.

Cosmetic cleansing agent in powder form according to one of the preceding points for the cleansing and/or care of human skin and/or human hair.

Cosmetic cleansing agent in powder form according to one of the preceding points, which is dissolved and foamed in contact with water.

Process for the preparation of a powdered cosmetic cleansing agent according to any one of the preceding points, in which all the ingredients are mixed and optionally sieved in a mixing apparatus for cosmetic powder products.

Cosmetic process for cleansing the human body and/or hair, in which a cosmetic cleansing agent in powder form according to one of the preceding points is dissolved in water, rubbed and/or foamed between the hands, distributed over the part of the body to be cleansed and rinsed off with water after a contact time of 10 seconds to 15 minutes.

Cosmetic use of a powdery cosmetic cleansing agent according to one of the preceding points for cleansing and/or care of the human body and/or hair, in particular for improvement of the
Untanglability,
Combability, especially on dry hair,
Skin and hair feel,
Clean feel, especially on dry skin and/or dry hair,
Hair volume,
Hairpin grip,
Suppleness of skin and/or hair.

Accordingly, a first object of the present invention is a cosmetic cleansing composition in powder form which - based on its total weight - has the following properties
a) 10.0 to 70.0 wt. % of at least one anionic surfactant,
b) 1.0 to 8.0% by weight of at least one C₂-C₆ polyhydric alcohol,
c) 20.0 to 40.0% by weight of at least one polysaccharide.

The previously defined powdered cosmetic cleansing agents offer the right properties for single-application portions, especially with regard to their dissolving and foaming behaviour during use. The high concentrations of active ingredients in the powders are associated with the advantages that fewer resources are consumed in the production and transport of the powders than with conventional, liquid cosmetic cleansers.

The powders according to the invention also have excellent cleansing and care properties, regardless of whether they are used on the skin or the hair or on the skin and the hair. In particular, important care aspects such as detangling, combability - especially on dry hair, skin and hair feel, clean feel—especially on dry skin and/or dry hair, hair volume, hair tip grip as well as suppleness of skin and/or hair could be increased or optimised by using the powders according to the invention.

The powder according to the invention comprises, as a first essential ingredient, from 10.0 to 70.0% by weight of at least one anionic surfactant a) (based on the total weight of the powder).

Anionic surfactants exhibit excellent foaming and/or cleaning behaviour, they are predominantly highly water-soluble and available in solid form.

Suitable anionic surfactant types for use in the powders of the invention include:
a) Ether carboxylic acids of the formula R—O—(CH₂-CH₂O)x—CH₂—COOH, in which R is a linear or is a branched, saturated or unsaturated alkyl group with 8 to 30 C atoms and =0 or 1 to 16,
b) Surfactants available from natural sources such as
   Acylglycinate,
   Acyl sarcosinate,
   Acyl lactylates,
   Acylglutamates,
   Acylaspartate,
   Acyltaurates,
   Acylisethionates,
   Sulphosuccinates and
      the corresponding salified forms of the above-mentioned surfactants, the acyl groups comprising from 10 to 30 carbon atoms.
c) Alpha-olefin sulphonates with 8 to 24 C atoms (alpha-olefin sulphonate surfactants),
d) Alkyl sulphates of the formula R—O—SO3X, in which R is preferably a straight or branched chain,
   is a saturated or unsaturated alkyl group with 8 to 30 C atoms and X is an alkali metal, alkaline earth metal, ammonium or alkanol amine ion,
e) Sulfonates of unsaturated fatty acids with 8 to 24 C-atoms and 1 to 6 double bonds,
f) Esters of tartaric acid and citric acid with alcohols which are addition products of about 2-15 molecules of ethylene oxide and/or propylene oxide to fatty alcohols with 8 to 22 C atoms, and/or
g) Alkyl and/or alkenyl ether phosphates of the formula

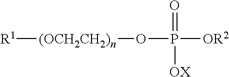

in which R¹ is preferably an aliphatic hydrocarbon radical having 8 to 30 carbon atoms, R² is hydrogen, a radical (CH₂CH₂O)ₙR¹ or X, n is numbers from 0 to 10 and X is hydrogen, an alkali metal or alkaline earth metal or the group —NR³R⁴R⁵R⁶, where R³ to R⁶ independently of one another are a C₁ to C₄ hydrocarbon radical.

Particularly preferred are surfactants from the aforementioned group b), as well as their corresponding salified forms, in which the acyl groups comprise 10 to 24 carbon atoms and in particular 10 to 18 carbon atoms.

Examples of particularly suitable surfactants from group b) are alkali metal, alkaline earth metal, ammonium and/or alkanolamine salts of so-called amino acid-based surfactants such as Cocoyl Glycinate, Cocoyl Sarcosinate, Lauroyl Sarcosinate, Myristoyl Sarcosinate, Oleyl Sarcosinate, Cocoyl Glutamate, Lauroyl Glutamate, stearoyl glutamate, lauroyl aspartate, palmitoyl aspartate, (C₁-C₄ alkyl) cocoyl taurate, (C₁-C₄ alkyl) lauroyl taurate, (C₁-C₄ alkyl) oleyl taurate and lauroyl lactylate, stearoyl lactylate, (C₁-C₄ alkyl) cocoyl isethionate, (C₁-C₄ alkyl) lauroyl isethionate, lauryl sulphosuccinate and/or any mixtures thereof.

Acylglutamates and/or acyl isethionates are particularly preferred.

Furthermore, surfactants from group d) are particularly preferred, because C₈-C₃₀ alkyl sulphates form a fine-pored, stable foam in combination with water even in small application quantities and are very well tolerated by the skin.

C₁₀-C₂₄ alkyl sulphates are highly preferred, and C₁₂-C₁₈ alkyl sulphates are particularly preferred, because these anionic surfactants, also known as “coco sulphates,” are obtained from natural coconut oil and are particularly suitable as ECOCERT-compliant ingredients for use in the powders of the invention.

Alkali salts such as potassium or sodium salts of coco sulphates are particularly preferred. Sodium Coco Sulphate (SCS) is particularly preferred.

In a preferred embodiment, the powders according to the invention contain—based on their total weight—15.0 to 65.0% by weight, preferably 20.0 to 60.0% by weight and particularly preferably 25.0 to 55.0% by weight of at least one anionic surfactant a).

Anionic surfactants from groups b) and d) have proved to be particularly effective for providing powders according to the invention which are particularly foam-strong and skin-compatible.

Amino acid-based surfactants, isethionates and/or alkyl sulphates are particularly preferred.

In a particularly preferred embodiment, powders according to the invention contain as anionic surfactant(s) a) amino acid-based surfactants, isethionates and/or alkyl sulphates.

Within this embodiment, acylglutamates, isethionates and/or alkyl sulphates are particularly preferred because they are mild and skin-friendly and they impart improved combability, smoothness, softness and shine to hair. Furthermore, acylglutamates, isethionates and/or alkyl sulphates can be produced on a purely vegetable basis and are also suitable for vegan cosmetics. Fatty acids of coconut oil can preferably serve as a raw material source.

In a further preferred embodiment, powders according to the invention contain—based on their total weight—.
ai) 10 to 60% by weight of at least one amino-acid-based surfactant, preferably an acyl glutamate and/or an isethionate and/or
aii) 0 to 60% by weight of at least one alkyl sulphate, preferably at least one compound known by the INCI name sodium coco sulphate.

These concentration ranges have proven to be particularly suitable. When working with these concentration ranges, the powders according to the invention have balanced properties in terms of solubility behaviour, application feel, cleaning and care performance.

The powder according to the invention comprises, as a second essential ingredient, from 1.0 to 8.0% by weight of at least one C₂-C₆ polyhydric alcohol b) (based on the total weight of the powder).

Suitable polyhydric C₂-C₆ alcohols b) improve and accelerate the (complete, residue-free) dissolution of the powder according to the invention in water, especially in the case where the powders according to the invention additionally contain water—insoluble care components (such as oils, waxes or perfumes). In addition, they are very well tolerated by the skin and improve the balance between cleansing and care in the powders.

In a preferred embodiment, the powders according to the invention contain—based on their total weight—1.0 to 7.5% by weight, preferably 1.5 to 7.0% by weight and particularly preferably 2.0 to 6.5% by weight of at least one polyhydric C₂-C₆ alcohol b).

Suitable polyhydric alcohols (b) include alditols such as mannitol, isomalt, lactitol, sorbitol and xylitol, threat, erythritol and arabitol, 1,2-propylene glycol, 1,3-butylene glycol, dipropylene glycol, glycerol and/or diglycerol. Preferred are 1.2-propylene glycol, 1,3-butylene glycol, dipropylene glycol, glycerol and/or diglycerol. Glycerine is particularly preferred.

In a further preferred embodiment, the powders according to the invention contain as polyhydric alcohol b) 1,2-propylene glycol, 1,3-butylene glycol, dipropylene glycol, glycerol and/or diglycerol.

Within this embodiment, powders according to the invention are particularly preferred which contain b) glycerol as polyhydric alcohol.

As a third essential ingredient, the powder according to the invention comprises 20.0 to 40.0% by weight of at least one polysaccharide (based on the total weight of the powder).

The polysaccharide or polysaccharides serve as binding and/or thickening agents in the compositions according to the invention. They come from natural sources, are preferably water-soluble or swellable in water and they support the formation of the desired external texture of the powder (compactness, low dusting, low stickiness).

In a preferred embodiment, the powders according to the invention contain—based on their total weight—21.0 to 38.0% by weight, preferably 22.0 to 36.0% by weight and particularly preferably 23.0 to 34.0% by weight of at least one polysaccharide c).

Polysaccharides c) suitable according to the invention are preferably composed of more than ten monosaccharide units and are available in powder form.

Preferred polysaccharides are
the celluloses and/or starches composed of glucose units and their derivatives and/or degradation products such as amylose, amylopectin and dextrins. Chemically and/or thermally modified starches, e.g. hydroxypropyl starch phosphate, dihydroxypropyl distarch phosphate or the commercial products Dry Flo®, are also advantageous according to the invention. Dextranes and their derivatives, e.g. dextran sulphate, are also preferred. Also preferred are non-ionic cellulose derivatives, such as methylcellulose, hydroxypropylcellulose, hydroxypropylmethylcellulose, hydroxyethylcellulose and/or carboxymethylcellulose;
the polysaccharides composed of fucose units, e.g. the commercial product Fucogel®;
the inulins composed of fructose units and a terminal glucose residue;
Heteroglycans such as alginic acid, carob gum, carrageenan, gum arabic, pectins, tragacanth, xanthan gum.

In a preferred embodiment, the powders according to the invention contain as polysaccharide c) starch fractions from maize, potatoes, rice, wheat and/or tapioca and/or derivatives of starch such as amylose, amylopectin and/or dextrin.

Within this embodiment, powders according to the invention are particularly preferred which contain starch fractions from maize as polysaccharide c).

The cleaning compositions in powder form according to the invention may comprise further ingredients which impart additional advantageous properties to them or support the advantageous properties of the cleaning compositions in powder form.

Such further ingredients may be selected, for example, from further surfactants and/or skin- and/or hair-conditioning active ingredients.

Other surfactants that can be considered are, for example, amphoteric, zwitterionic and/or nonionic surfactants.

Suitable amphoteric and/or zwitterionic surfactants in the sense of the present invention are Alkyl betaines, alkylamidoalkyl betaines, alkylamphoacetates, alkylamphodiacetates, alkylamphopropionates, alkylamphodipropionates, alkylsultaines, alkylhydroxysultaines, alkylamine oxides, alkylamphoglycinates, alkyliminodiacetates, alkyliminodipropionates, alkylamphopropylsulphonates, alkylamphocarboxyglycinates and alkylamphocarboxypropionates.

Suitable alkyl betaines and/or alkylamidopropyl betaines preferably comprise C₄-C₂₄, more preferably C₆-C₁₈, particularly preferably C₈-C₁₄ alkyl chains, which may be linear or branched, linear chains being preferred.

Cocamidopropyl betaine is particularly preferred.

Particularly suitable alkylamphodiacetates, alkylamphodiacetates, alkylamphopropionates or alkylamphodipropionates are sodium cocoamphodiacetate and disodium cocooamphodiacetate.

In a preferred embodiment, the cleansing compositions in powder form according to the invention additionally contain at least one amphoteric and/or zwitterionic surfactant in a proportion by weight of 0.5 to 10% by weight (more preferably 1.0 to 9.0% by weight, particularly preferably 1.5 to 8.0% by weight and especially 2.0 to 7.0% by weight) of the total weight of the cosmetic cleansing composition.

Suitable non-ionic surfactants in the sense of the present invention are:
Amine oxides, for example the surfactants known under the INCI names Cocamine Oxide, Lauramine Oxide and/or Cocamidopropylamine Oxide and commercially available from various suppliers,
Fatty acid alkanolamides, in particular the compounds known under the INCI designation Cocoamide MEA,
Fatty alcohol alkoxylates (especially ethoxylates) with a C-chain length of 8 to 24, especially 10 to 20, and a degree of alkoxylation (ethoxylation degree) of 2 to 30, such as laureth-4,
Mixtures of alkyl(oligo)glucosides and fatty alcohols, for example, the commercially available product Montanov®68,
Alkyl(oligo)glycoside. Suitable alkyl (oligo)glycosides can be selected from compounds of the general formula of RO-[G]ₓ, in which [G] is preferably derived from aldoses and/or ketosis with 5-6 carbon atoms, preferably from glucose.

The index number x stands for the degree of oligomerization (DP), i.e. the distribution of mono- and oligoglycosides. The index number x preferably has a value in the range from 1 to 10, particularly preferably in the range from 1 to 3, whereby it may not be an integer but a fractional number that can be determined analytically.

Particularly preferred alkyl (oligo)glycosides have a degree of oligomerization between 1.2 and 1.5. The radical R preferably represents at least one alkyl and/or alkenyl radical containing 4 to 24 carbon atoms.

Especially preferred alkyl (oligo)glycosides are the compounds known under the INCI designations Caprylyl/Capryl Glucosides, Decyl Glucosides, Lauryl Glucosides and Coco Glucosides.

The nonionic surfactant(s) may be used in the cosmetic cleansing compositions according to the invention in powder form (based on their total weight) preferably in an amount of from 0 to 3.0% by weight, more preferably from 0.1 to 2.50% by weight, particularly preferably from 0.2 to 2.25% by weight and especially from 0.25 to 2.00% by weight.

Among suitable skin- and/or hair-conditioning active ingredients mentioned above, the following are preferably included
cationic polymers, especially cationic polysaccharide polymers,
Trimethylglycine,
Lipids,
vegetable oils, butters and/or waxes,
protein hydrolysates,
Silicones,
Plant extracts and/or
Understanding anti-dandruff active ingredients.

Suitable cationic polymers in the sense of the present invention include preferably cationic polymers of natural origin, in particular cationic polymers of natural origin such as cationic polysaccharide polymers. Particularly suitable examples are:
quaternized cellulose derivatives, as referred to under the names Celquat® and Polymer JR® are available on the market,
cationic starch derivatives, such as those commercially available under the name Mirustyle®,
hydrophobically modified cellulose derivatives, such as the cationic polymers sold under the trade name SoftCat®,
cationic alkyl polyglycosides,
cationized honey, for example the commercial product Honeyquat® 50,
cationic guar derivatives, in particular the products marketed under the trade names Cosmedia® Guar N-Hance® and Jaguar®,
cationic cassia and/or inulin derivatives such as those listed under the INCI designation Cassia,
Hydroxypropyltrimonium Chloride and Hydroxypropyltrimonium Inulin known cationic polymers.

The cosmetic compositions in powder form according to the invention may preferably contain cationic polymers in a proportion by weight of 0 to 4%, more preferably 0.1 to 3.0%, and in particular 0.15 to 2.0%, of the total weight of the compositions.

In a preferred embodiment, cleansing compositions in powder form according to the invention additionally contain at least one cationic polymer in a proportion by weight of 0.1 to 3% by weight of the total weight of the cosmetic cleansing composition.

Within this embodiment, it is particularly preferred if the cationic polymer is selected from cationic polysaccharide polymers and in particular from cationic polymers obtainable from cellulose, guar, cassia and/or inulin.

Trimethylglycine may be used in the powder compositions according to the invention in a proportion by weight of 0.1 to 3% of the total weight of the powder.

Trimethylglycine in combination with the surfactants a) has a foam-stabilising and irritation-reducing effect. Moreover, a content of trimethylglycine in the powder compositions according to the invention leads to a significant conditioning benefit (improvement of skin hydration, improvement of (wet) combability and/or strengthening of hair fibres).

In a preferred embodiment, cosmetic cleansing compositions according to the invention in powder form additionally contain trimethylglycine (betaines) in a proportion by weight of 0.1 to 3% by weight of the total weight of the cleansing composition.

In a further preferred embodiment, powdered cosmetic cleansing compositions according to the invention contain at least one further cosmetic active ingredient and/or excipient selected from the group consisting of
Lipids,
vegetable oils, butters and/or waxes,
Protein hydrolysates,
Silicones,
Plant extracts and/or
Anti-dandruff active ingredients.

Suitable lipids in the sense of the present invention are understood to be all fats and fat-like substances which melt preferably in the range of 30-150° C. These include triglycerides, mono- and/or diglycerides, waxes, fatty and wax alcohols, fatty acids, esters and/or ethers of fatty alcohols and fatty acids as well as fatty acid amides, hydrocarbons, lipoproteins, glycolipids, phospholipids or any mixtures of these substances.

Triglycerides are understood to be “natural” oils, preferably vegetable oils.

These preferably include triglycerides and mixtures of triglycerides such as amaranth seed oil, apricot kernel oil, argan oil, avocado oil, babassu oil, bamboo oil, baobab oil, canola oil, safflower oil, pomegranate seed oil, rosehip seed oil, hemp oil, hazelnut oil, red currant oil, black currant oil, jojoba oil, coconut oil, pumpkin seed oil, Macadamia nut oil, mallow oil, (sweet) almond oil, mango kernel oil, evening primrose oil, olive oil, Brazil nut oil, passion fruit oil, palm kernel oil, peach kernel oil, rambutan oil, rice bran oil, Castor Oil, Safflower Oil, Sasanqua Oil, Sesame Oil, Soybean Oil, Sunflower Oil, Tea Tree Oil, Grape Seed Oil, Tsubaki Oil, Walnut Oil, Wheat Germ Oil and/or Meadowfoam Oil.

Suitable plant butters preferably include shea butter, mango butter, murumuru butter, cocoa butter, apricot kernel butter, mafuri butter, bacuri butter, tucuma butter, ucuuba butter and/or cupuacu butter.

Waxes are natural or artificially obtained substances with the following properties: they are of solid to brittle-hard consistency, coarse to fine crystalline, translucent to cloudy, and they melt above 30° C. without decomposition. They are already low viscosity and non-fibrous a little above the melting point and show a strongly temperature-dependent consistency and solubility. Natural vegetable waxes with a melting point of 30-150° C., for example, can be used according to the invention, such as e.g. Candelilla wax, carnauba wax, Japan wax, esparto grass wax, cork wax, guaruma wax, rice germ oil wax, sugar cane wax, ouricury wax, montan wax, sunflower wax, fruit waxes such as orange waxes, lemon waxes, grapefruit wax, bayberry wax and animal waxes such as beeswax, shellac wax, spermaceti, lanolin and brushing fat. In the spirit of the invention, it may be advantageous to use hydrogenated or cured waxes. Natural waxes that can be used according to the invention also include mineral waxes, such as ceresin and ozokerite, or petrochemical waxes, such as petrolatum, paraffin waxes and microwaxes. Chemically modified waxes, in particular the hard waxes, such as montan ester waxes, sasol waxes and hydrogenated jojoba waxes, can also be used as wax components. Synthetic waxes which can be used according to the invention include, for example, waxy polyalkylene waxes and polyethylene glycol waxes.

Glyceride mixtures such as Cutina HR (hardened castor oil) and Novata AB (mixture of C₁₂-C₁₈ mono-, di- and triglycerides) are preferably used as mono- and/or diglycerides.

The fatty alcohols which can be used according to the invention include e.g. the unbranched C₁₂-C₅₀ fatty alcohols, in particular the C₁₄-C₃₀ fatty alcohols, which are obtained from natural fats, oils and waxes, such as lauryl alcohol, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. Preferred according to the invention are unbranched, saturated and unsubstituted fatty alcohols.

However, branched, saturated or unsaturated fatty alcohols can also be used according to the invention. Also usable according to the invention are fatty alcohol cuts, such as are produced during the reduction of naturally occurring fats and oils.

Preferred fatty acids are C₁₂-C₄₀ fatty acids or mixtures thereof. These include, for example, lauric, tridecanic, myristic, pentadecanic, palmitic, margaric, stearic, nonadecanic, arachic, behenic, lignoceric, cerotinic, melissic, erucic and elaeostearic acids, as well as substituted fatty acids such as 12-hydroxystearic acid. Such as 12-hydroxystearic acid, and the amides or monoethanolamides of the fatty acids, this list being exemplary and not restrictive.

Suitable hydrocarbons are understood to be liquid or solid hydrocarbons such as petrolatum, ceresin, ozokerite, paraffins, isoparaffins and/or squalene.

Suitable glycolipids in the sense of the present invention are sugar surfactants, alkyl polyglycosides and/or ceramides.

A suitable phospholipid in the sense of the present invention is lecithin.

Lipids can be used in the cleaning compositions according to the invention in powder form, preferably in a proportion by weight of 0.01 to 10% by weight of the total weight of the cleaning composition. Preferred amounts are from 0.05 to 8.5% by weight and particularly preferred from 0.1 to 7% by weight.

In a preferred embodiment, the cleaning compositions according to the invention in powder form contain—based on their total weight—0.01 to 10% by weight of at least one lipid, preferably selected from vegetable oils, butters and/or waxes, ceramides, lecithin and/or fatty acids.

Suitable protein hydrolysates are product mixtures that can be obtained by acidic, basic or enzymatic catalyzed degradation of proteins.

Protein hydrolysates of vegetable, animal and/or marine origin can be used.

Animal protein hydrolysates include elastin, collagen, keratin, silk and milk protein hydrolysates, which may also be in the form of salts. Such products are marketed under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolysates of vegetable origin are preferred, e.g. soy, almond, rice, pea, potato and wheat protein hydrolysates. Such products are available under the trademarks Gluadin® (Cognis), DiaMin® (Diamalt), Lexein® (Inolex) and Crotein® (Croda).

Also applicable are cationized protein hydrolysates, whereby the underlying protein hydrolysate can originate from the animal, for example from collagen, milk or keratin; from the plant, for example, from wheat, maize, rice, potatoes, soya or almonds; from marine life forms, for example from fish collages or algae; or from biotechnological protein hydrolysates. The protein hydrolysates underlying the cationic derivatives can be obtained from the corresponding proteins by a chemical, in particular alkaline or acid hydrolysis, by an enzymatic hydrolysis and/or a combination of both types of hydrolysis. The hydrolysis of proteins results in protein hydrolysate with a molecular weight distribution of about 100 daltons to several thousand daltons. Preferred are such cationic protein hydrolysates whose underlying protein portion has a molecular weight of 100 up to 25000 Daltons, preferably 250 to 5000 daltons.

Furthermore, under cationic protein hydrolysates quaternized amino acids and mixtures thereof. The quaternisation of the protein hydrolysates or of the amino acids is often carried out by means of quaternary ammonium salts such as N,N-dime thyl-N-(n-alkyl)-N-(2-hydroxy-3-chloro-n-propyl)-ammonium halides. Furthermore the cationic protein hydrolysates can also be further derivatised. Typical examples of cationic protein hydrolysates and derivatives are the products known under the INCI- designation and commercially available: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed rice protein, Cocodimonium Hydroxypropyl Hydrolysed silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) in the total weight of the powdered cleaning compositions according to the invention is preferably 0.01 to 5% by weight, more preferably 0.025 to 3% by weight and in particular 0.05 to 2% by weight.

In a preferred embodiment, the cleaning compositions according to the invention in powder form contain—based on their total weight—0.01 to 5% by weight of at least one protein hydrolysate.

Suitable silicones in the sense of the present invention may be selected from:
(i) Polyalkylsiloxanes, polyarylsiloxanes, polyalkylarylsiloxanes which are volatile or non-volatile, straight chain, branched or cyclic, cross-linked or non-cross-linked;
(ii) polysiloxanes containing in their general structure one or more organofunctional groups selected from:
   (a) substituted or unsubstituted aminated groups;
   (b) (per)fluorinated groups;
   (c) thiol groups;
   (d) carboxylate groups;
   (e) hydroxylated groups;
   (f) alkoxylated groups;
   (g) acyloxyalkyl groups;
   (h) amphoteric groups;
   (i) bisulphite groups;
   (j) hydroxyacylamino groups;
   (k) carboxy groups;
   (l) sulphonic acid groups; and
   (m) sulphate or thiosulphate groups;
(iii) linear polysiloxane (A)-polyoxyalkylene (B) block copoylmers of the type (A-B)n with n>3;
(iv) grafted silicone polymers with a non-silicone organic backbone consisting of an organic backbone formed from non-silicone organic monomers onto which at least one polysiloxane macromer has been grafted in the backbone and optionally at least one end of the backbone;
(v) grafted silicone polymers having a polysiloxane backbone onto which have been grafted non-silicone organic monomers comprising a polysiloxane backbone onto which has been grafted, in the chain and optionally at least one of its ends, at least one non-silicone organic macromer;
(vi) or mixtures thereof.

The proportion by weight of the silicone(s) in the total weight of the powdered cleaning compositions according to the invention is preferably 0.01 to 5% by weight, more preferably 0.025 to 3% by weight and in particular 0.05 to 2% by weight.

In a preferred embodiment, the cleaning compositions according to the invention in powder form contain—based on their total weight—0.01 to 5% by weight of at least one silicone.

Suitable plant extracts are mainly the extracts of green tea, oak bark, nettle, witch hazel, hops, henna, chamomile, burdock root, horsetail, hawthorn, linden blossom, almond, Aloe vera, spruce needle, horse chestnut, date palm, cinnamon tree, sandalwood, juniper, coconut, mango, apricot, lime, wheat, kiwi, melon, orange, grapefruit, sage, rosemary, birch, mallow, Valerian, Meadowfoam, Quender, Yarrow, Thyme, Melissa, Hauhechel, Coltsfoot, Marshmallow, Meristem, Ginseng, Coffee, Cocoa, Moringa, Ginger Root and Ayurvedic plant extracts such as Aegle Marmelos (Bilwa), Cyperus Rotundus (Nagar Motha), Emblica Officinalis (Amalki), Morida Citrifolia (Ashyuka), Tinospora Cordifolia (Guduchi), Santalum album, (Chandana), Crocus Sativus (Kumkuma), Cinnamonum Zeylanicum and Nelumbo Nucifera (Kamala), sweet grasses such as wheat, barley, rye, oats, spelt, corn, the different varieties of millet (panicle millet, Finger millet, foxtail millet as examples), sugarcane, ryegrass, meadow foxtail, smooth oats, bunchgrass, meadow fescue, pipegrass, bamboo, cotton grass, lampbush grasses, Andropogonodeae (Imperata Cylindrica also called flame grass or cogon grass), buffalo grass, silt grasses, dogtooth grasses, love grasses, Cymbopogon (lemon grass), Oryzeae (rice), Zizania (wild rice), Beach oats, perennial oats, honey grasses, quaking grasses, meadow grasses, couch grasses, and Echinacea, especially Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench and Echinacea pallida (Nutt), all kinds of seaweeds, coral moss and seaweed (such as Macrocystis Pyrifera extract), all kinds of vines and pericarp of Litchi chinensis.

Suitable extracts can be obtained from the fruits, seeds, flowers, roots, leaves and/or barks of the above plants or from the whole plants (algae, seaweed).

According to the invention, the plant extracts can be used both in pure and diluted form. If they are used in diluted form, they usually contain approx. 2-80% by weight of active substance and as solvent the extracting agent or extracting agent mixture used in their extraction. Suitable extraction agents are usually water and/or alcohols.

The plant extract(s) may be used in the powdered cleansing compositions according to the invention preferably in total amounts of from 0.001 to 1% by weight, preferably from 0.0025 to 0.75% by weight and more preferably from 0.05 to 0.5% by weight, the amounts being based on the weight of the cleansing compositions.

In a preferred embodiment, the cleaning compositions according to the invention in powder form contain—based on their total weight—0.01 to 5% by weight of at least one plant extract.

For the purposes of the present invention, suitable anti-dandruff agents include piroctone olamine, climbazole, zinc pyrithione, ketoconazole, salicylic acid, sulphur, selenium sulphide, tar preparations, undecenic acid derivatives, burdock root extracts, poplar extracts, nettle extracts, walnut shell extracts, birch extracts, willow bark extracts, rosemary extracts, arnica extracts and/or propanediol caprylates.

Preferred for use in the cosmetic powdered cleansing compositions according to the invention are natural anti-dandruff active ingredients such as the aforementioned plant extracts and/or anti-dandruff active ingredients accessible from natural sources such as propanediol caprylates.

The proportion by weight of antidandruff agents in the total weight of the cosmetic powdered cosmetic powdery cleansing compositions according to the invention is preferably from 0.01 to 5 wt. %, more preferably 0.025 to 4 wt. % and particularly preferably 0.05 to 3 wt. %.

In a preferred embodiment, the cleansing compositions according to the invention in powder form contain—based on their total weight—0.01 to 5% by weight of at least one anti-dandruff active ingredient, preferably a natural anti-dandruff active ingredient and particularly preferably propanediol caprylates.

Further active ingredients, auxiliary substances and additives which may preferably be contained in the powdered cosmetic cleansing compositions according to the invention are, for example:
Vitamins, provitamins and vitamin precursors
cationic surfactants,
abrasives,
Perfumes, if necessary also in capsule form,
UV-Filter,
(natural) dyes for colouring the agent,
Active ingredients such as bisabolol and/or allantoin,
Complexing agents such as EDTA, NTA, β-alanine diacetic acid and phosphonic acids,
Antioxidants,
pH adjusting agents such as citric acid and/or lactic acid,
Salts (NaCl).

A significant advantage of the powdered cleaning compositions according to the invention over commercially available concentrates is that no free water needs to be added to them, which means that they have an even smaller volume and preservation is not absolutely necessary.

A small amount of water may nevertheless be present in the powdered cleaning compositions of the invention as a component of commercial products, but preferably they are substantially free of water.

For the purposes of the present invention, “substantially free” of water is preferably understood to mean a maximum water content of 5% by weight, more preferably 3% by weight and particularly preferably 1.5% by weight (based on the total weight of the powdered cleaning compositions according to the invention).

Similarly, it is possible that the powdered cleaning compositions according to the invention nevertheless comprise a small proportion of preservatives as a component of commercial products, but preferably they are substantially free of preservatives.

For the purposes of the present invention, “substantially free” of preservatives is preferably understood to mean a maximum preservative content of 1% by weight, more preferably of 0.5% by weight and particularly preferably of 0.1% by weight (based on the total weight of the powdered cleaning compositions according to the invention).

In a preferred embodiment, the powdered cosmetic cleansing compositions of the invention are substantially free of water and/or preservatives.

The invention also relates to a powdered cosmetic cleansing composition as previously described for the cleansing and/or care of human skin and/or human hair.

It is beneficial and easy for the user to be able to use one and the same product for skin and hair. A composition that is suitable for both due to its formulation and packaging can therefore be considered advantageous.

The invention also relates to a powdered cosmetic cleansing composition as previously described in the form of a powder which dissolves and foams on contact with water.

A powder form is well, namely particularly quickly, soluble. This corresponds to the spirit of the times, but at the same time is associated with a small “wellness experience” during application, as the use of the powder creates a new feeling of application that differs from previous experiences and emphasises the playful side of body care.

The invention also relates to a process for the preparation of a powder cosmetic cleansing composition as previously described, in which all the ingredients are mixed and optionally sieved in a mixing apparatus for powder cosmetic products.

This has the advantage of providing a solid cosmetic cleansing composition in powder form, and the sieving ensures a uniform granulation that is perceived as pleasant. The advantage of such a process is that it does not require melting at high temperatures, which saves energy and is very gentle on the ingredients. This is particularly important when using largely natural, low-processed ingredients that can provide benefits not achievable with highly processed products.

The invention also relates to a process for cleansing the human body and/or hair, in which a cosmetic cleansing agent in powder form, as previously described, is dissolved in water, rubbed and/or foamed between the hands, distributed over the part of the body to be cleansed and rinsed off with water after a contact time of 10 seconds to 15 minutes.

This procedure makes the advantages described above tangible for a user and thus represents an enrichment not only for personal hygiene and care but also a sensory enrichment.

The invention also relates to the use of a cosmetic cleansing composition in powder form, as previously described, for cleansing and/or caring for the human body and/or hair, in particular for improving the
Untanglability,
Combability, especially on dry hair,
Skin and hair feel,
Clean feel, especially on dry skin and/or dry hair,
Hair volume,
Hairpin grip,
Suppleness of skin and/or hair.

This use makes the advantages described above tangible for a user and thus represents an enrichment not only for personal hygiene and care but also a sensory enrichment.

Another novel form of application of the powder compositions according to the invention, which is associated with a special experience, is packaging, for example in a (paper) sachet or a jar. In addition to the new sensory experience, this is accompanied above all by the advantage of a relatively fast resolution due to a high surface area. The packaging materials glass and paper are easily recyclable packaging materials that are popular with environmentally conscious buyers, and their properties are therefore well suited to the properties of the powdered cosmetic compositions described and claimed here.

It is also possible to fill the powder compositions according to the invention into a jar, for example made of glass. Since the strength of these compositions is in a range that allows an application portion to be taken manually, without a tool, from a crucible intended for multiple applications. If the composition has been foamed in a crucible during its manufacture, the result is a particularly interesting feeling.

### EXAMPLES

Basic exemplary compositions are shown in the following Tables 1-7 (compositions Z1 to Z25). Quantities refer to [% by weight] (unless otherwise stated):

TABLE 1

| Ingredient | Z1 | Z2 | Z3 | Z4 |
|---|---|---|---|---|
| (a): one or more anionic | 10-70 | 15-65 | 20-60 | 25-55 |
| surfactant(s) | | | | |
| b): polyhydric C₂-C₄ alcohol | 1-8 | 1-7.5 | 1.5-7 | 2-6.5 |
| c): Polysaccharide | 20-40 | 21-38 | 22-36 | 23-34 |
| If necessary, other auxiliaries | ad 100 | ad 100 | ad 100 | ad 100 |
| and additives | | | | |

TABLE 2

| Ingredient | Z5 | Z6 | Z7 | Z8 |
|---|---|---|---|---|
| (a): one or more anionic | 10-70 | 15-65 | 20-60 | 25-55 |
| surfactant(s) | | | | |
| b): polyhydric C₂-C₄ alcohol | 1-8 | 1-7.5 | 1.5-7 | 2-6.5 |
| c): Polysaccharide | 20-40 | 21-38 | 22-36 | 23-34 |
| amphoteric and/or zwitterionic | 0.5-10 | 1-9 | 1.5-8 | 2-7 |
| surfactant | | | | |
| If necessary, other auxiliaries | ad 100 | ad 100 | ad 100 | ad 100 |
| and additives | | | | |

TABLE 3

| Ingredient | Z9 | Z10 | Z11 | Z12 |
|---|---|---|---|---|
| (a): one or more anionic | 10-70 | 15-65 | 20-60 | 25-55 |
| surfactant(s) | | | | |
| b): polyhydric C₂-C₄ alcohol | 1-8 | 1-7.5 | 1.5-7 | 2-6.5 |
| c): Polysaccharide | 20-40 | 21-38 | 22-36 | 23-34 |
| cationic polymer | 0.1-3 | 0.1-2 | 0.15-2 | 0.2-1.5 |
| If necessary, other auxiliaries and | ad 100 | ad 100 | ad 100 | ad 100 |
| additives | | | | |

TABLE 4

| Ingredient | Z13 | Z14 | Z15 | Z16 |
|---|---|---|---|---|
| (a): one or more anionic | 10-70 | 15-65 | 20-60 | 25-55 |
| surfactant(s) | | | | |
| b): polyhydric C₂-C₄ alcohol | 1-8 | 1-7.5 | 1.5-7 | 2-6.5 |
| c): Polysaccharide | 20-40 | 21-38 | 22-36 | 23-34 |
| Trimethylglycine | 0.1-3 | 0.15-2.5 | 0.2-2 | 0.25-1.5 |
| If necessary, other auxiliaries | ad 100 | ad 100 | ad 100 | ad 100 |
| and additives | | | | |

TABLE 5

| Ingredient | Z17 | Z18 | Z19 | Z20 |
|---|---|---|---|---|
| (a): one or more anionic | 10-70 | 15-65 | 20-60 | 25-55 |
| surfactant(s) | | | | |
| b): polyhydric C₂-C₄ alcohol | 1-8 | 1-7.5 | 1.5-7 | 2-6.5 |
| c): Polysaccharide | 20-40 | 21-38 | 22-36 | 23-34 |
| Lipids, vegetable oils, butters | 0.01-10 | 0.05-9 | 0.1-8.5 | 0.15-8 |
| and/or waxes, protein | | | | |
| hydrolysates, silicones, plant | | | | |
| extracts and/or anti-dandruff | | | | |
| active ingredients | | | | |
| If necessary, other auxiliaries | ad 100 | ad 100 | ad 100 | ad 100 |
| and additives | | | | |

TABLE 6

| Ingredient | Z21 | Z22 | Z23 | Z24 |
|---|---|---|---|---|
| a): Amino acid-based surfactant, | 10-70 | 15-65 | 20-60 | 25-55 |
| isethionate and/or alkyl sulphate | | | | |
| b): 1.2-propylene glycol, 1.3-butylene | 1-8 | 1-7.5 | 1.5-7 | 2-6.5 |
| glycol, dipropylene glycol, glycerol | | | | |
| and/or diglycerol | | | | |
| c): Starch fractions and/or derivatives | 20-40 | 21-38 | 22-36 | 23-34 |
| of starch | | | | |
| If necessary, other auxiliaries and | ad 100 | ad 100 | ad 100 | ad 100 |
| additives | | | | |

TABLE 7

| Ingredient | Z25 |
|---|---|
| Sodium Cocoyl Isethionate, (Di)sodium Cocoyl Glutamate | 45-55 |
| Glycerine | 3-7 |
| Maize Starch (ZEA Mays (Corn) Starch) | 22-34 |
| Cocamidopropyl betaine | 0-7 |
| Guar Hydroxypropyltrimonium Chloride | 0-1.5 |
| Trimethylglycine (betaine) | 0-1.5 |
| If necessary, other auxiliaries and additives | ad 100 |

What is claimed is:

1. A cosmetic cleansing composition in powder form comprising, based on the total weight of the cleansing composition
a) 10.0 to 70.0 wt. % of at least one anionic surfactant,
b) 1.0 to 8.0% by weight of at least one C₂-C₆ polyhydric alcohol, and
c) 20.0 to 40.0% by weight of at least one polysaccharide.

2. The cosmetic cleansing composition in powder form according to claim 1, wherein the anionic surfactant(s) a) is selected from the group consisting of amino acid-based surfactants, isethionates, alkyl sulphates, and combinations thereof.

3. The cosmetic cleansing composition in powder form according to claim 1, wherein the polyhydric alcohol b) is selected from the group consisting of 1,2-propylene glycol, 1,3-butylene glycol, dipropylene glycol, glycerol, diglycerol, and combinations thereof.

4. The cosmetic cleansing composition in powder form according to claim 1, wherein the polysaccharide c) is selected from the group consisting of starch fractions from maize, potatoes, rice, wheat, tapioca, derivatives of starch, and combinations thereof.

5. The cosmetic cleansing composition in powder form according to claim 1, further comprising
at least one amphoteric and/or zwitterionic surfactant in a weight proportion of 0.5 to 10% by weight of the total weight of the cosmetic cleaning composition and/or
at least one cationic polymer in a weight proportion of 0.1 to 3% by weight of the total weight of the cosmetic cleaning composition and/or
Trimethylglycine (betaine) in a weight proportion of 0.1 to 3% by weight of the total weight of the cosmetic cleaning composition.

6. The cosmetic cleansing composition in powder form according to claim 1, further comprising at least one cosmetic active ingredient and/or excipient selected from the group consisting of
Lipids,
vegetable oils, butters and/or waxes,
Protein hydrolysates,
Silicones,
Plant extracts,
Anti-dandruff active ingredients, and
combinations thereof.

7. The cosmetic cleansing composition in powder form according to claim 1, which is substantially free of water and/or preservatives.

8. The cosmetic cleansing composition in powder form according to claim 1, which is dissolved and foamed in contact with water.

9. A cosmetic process for cleansing the human body and/or hair, comprising obtaining the cosmetic cleansing composition in powder form according to claim 1, dissolving the composition in water, rubbing and/or foaming between the hands, and distributing over the part of the body to be cleansed and rinsing off with water after a contact time of 10 seconds to 15 minutes.

10. The cosmetic cleansing composition in powder form according to claim 4, wherein the polysaccharide c) starch derivatives are selected from the group consisting of amylose, amylopectin and/or dextrin.
